(19) United States
(12) Patent Application Publication
(10) Pub. No.: US 2021/0340480 A1
(43) Pub. Date: Nov. 4, 2021

(54) PROCESS FOR PRODUCING ALCOHOLS WITH CLOSTRIDIUM ON A SOLID SUPPORT

(71) Applicant: IFP Energies nouvelles, Rueil-Malmaison Cedex (FR)

(72) Inventors: Helena GONZALEZ PENAS, Rueil-Malmaison Cedex (FR); Marcel ROPARS, Rueil-Malmaison Cedex (FR); Eszter TOTH, Rueil-Malmaison Cedex (FR); Vincent COUPARD, Rueil-Malmaison Cedex (FR); Sandra MENIR, Rueil-Malmaison Cedex (FR); Nicolas LOPES FERREIRA, Rueil-Malmaison Cedex (US)

(73) Assignee: IFP Energies nouvelles, Rueil-Malmaison Cedex (FR)

(21) Appl. No.: 17/279,769

(22) PCT Filed: Sep. 26, 2019
(86) PCT No.: PCT/EP2019/075972

§ 371 Date: Mar. 25, 2021

(30) Foreign Application Priority Data
Sep. 28, 2018 (FR) 1871106

(51) Int. Cl. C12M 1/12; C12P 7/06; C12P 7/16; C12P 7/28; C12P 7/04; C12N 11/093

(52) U.S. Cl. CPC C12M 25/00; C12P 7/065; C12N 11/093; C12P 7/28; C12P 7/04; C12P 7/16

(57) ABSTRACT

The present invention relates to a process for producing alcohols, in which a sugary fluid is introduced into a fermentation reactor (2) to produce a fermentation must enriched in isopropanol, butanol, ethanol and acetone relative to the sugary fluid, the fermentation reactor (2) comprising a biomass produced by a strain belonging to the genus Clostridium which is supported on a solid support (9) comprising a polyurethane foam; and to a fermentation reactor (2) comprising said biomass supported on said solid support (9).

### TECHNICAL FIELD

The present description relates to a process for producing alcohols by fermentation of a sugary fluid.

### PRIOR ART

In order to meet the energy transition challenges, considerable research is being conducted to develop “green” processes, affording access to chemical intermediates in an alternative manner to the refining of petroleum and/or petrochemistry.

Alcohols derived from fermentation processes (e.g. isopropanol and n-butanol) are among the most promising replacements for petrochemical derivatives. ABE (Acetone-Butanol-Ethanol) fermentation, performed by microorganisms belonging to the genus Clostridium, is one of the oldest fermentations to have been industrialized (at the start of the 20th century) and has since been extensively studied. More recently, IBE (Isopropanol-Butanol-Ethanol) fermentation, producing a mixture of isopropanol, butanol and ethanol and also performed by microorganisms belonging to the genus Clostridium, has been the subject of numerous studies.

As regards the fermentation approach employed in this type of process, batch production remains the conventional method for ABE and IBE fermentations, despite the low productivity displayed for this type of process, in the range 0.1-0.7 g/L.h (see, for example, Jones D. T., Woods D. R., 1986, Acetone-Butanol Fermentation Revisited. Microbiol. Rew., 50 (4), 484-524 or Table 16.6 Lopez-Contreras A. et al. chapter book 16, Bioalcohol Production: Biochemical Conversion of Lignocellulosic Biomass, 2010). However, these productivities remain too low to envisage an economically viable industrial process.

A continuous process with cells in suspension in a homogeneous reactor may also be envisaged. However, the productivity is also relatively low and cannot easily be significantly increased. One of the major technical problems is the concentration of the cells in the fermentation medium, which is mainly controlled by the dilution rate applied in the process. This rate cannot be high, to avoid cell “wash-out” in the fermenter. For these reasons, strong interest has been shown in recent years in methods directed toward high retention of the microbial biomass. Two means exist: “immobilization of the cells” and cell “recycling” with retention by means of filter membranes.

Two immobilization techniques for the continuous process are often cited: adsorption on a solid support and confinement, the two techniques having been studied in the literature for the production of ABE.

In the first case of adsorption on a solid support, the physical adsorption of microorganisms onto a solid surface takes place via electrostatic forces, van der Waals forces, or by covalent bonding between the bacterial cell membrane and the support. As there is no physical barrier between the microbial biofilm and the fermentation solution, various equilibria between the degrees of adsorption, of cell detachment and of recolonization of the solid support may be achieved as a function of the solid support, of the implementation and of the operating conditions. It should be noted that the immobilized cells are typically surrounded with polysaccharides excreted by the microorganisms themselves (EPS: “Extracellular Polymeric Substances”), and have different growth and bioactivity regimes only when the cells are in suspension (see, for example, Halan B., Buehler K., Schmid A., 2012, Biofilms as living catalysts in continuous chemical syntheses, Trends in Biotechnol., 30 (9), 453-465).

Several solid supports have been tested and prove to be advantageous according to the literature for the fermentation of ABE type, including charcoal (see, for example, Qureshi N., Maddox I. S., 1987, Continuous solvent production from whey permeate using cells of Clostridium acetobutylicum immobilized by adsorption onto bonechar, Enzyme Microb. Technol., (9), 668-371), bricks (see, for example, Qureshi N., Schripsema J., Lienhardt J., Blaschek H. P., 2000, Continuous solvent production by Clostridium beijerinckii BA101 immobilized by adsorption onto brick, World Journal of Microbiology & Biotechnology, (16), 377-382), and paper pulp (see, for example, Survase S. A., van Heiningen A., Granstrom T., 2012, Continuous bio-catalytic conversion of sugar mixture to acetone-butanol-ethanol by immobilized Clostridium acetobutylicum DSM 792, Appl. Microbiol. Biotechnol., (93), 2309-2316). On the other hand, such solid supports are not synthetic and give rise to major problems of reproducibility for fermentation processes.

In the case of encapsulation (immobilization by confinement), the microorganisms are introduced inside a porous matrix, so as to avoid their diffusion into the external medium, while at the same time allowing the transfer of material for the substrate and the nutrients, and also the reaction products. Examples of supports using the encapsulation confinement technique include alginate beads (see, for example, Mollah A. H., Stuckey D. C., 1993, Maximizing the production of acetone-butanol in alginate bead fluidized bed reactor using Clostridium acetobutylicum, J. Chem. Tech. Biotechnol., (56), 83-89), and k-carrageenan (see, for example, Godia F., Howard I., Scott D., Davison B. H., 1990, Use of immobilized microbial membrane fragments to remove oxygen and favor the acetone-butanol fermentation, Biotechnol. Prog., 1990, 210-213).

### SUMMARY OF THE INVENTION

A first object of the present description is to provide a process for fermentation of IBEA (Isopropanol-Butanol-Ethanol-Acetone) type in a reactor in which the hydraulic dilution rate is different from the dilution rate of the active biomass. To this end, a process that is capable of at least partly fixing the bacterial biomass by adsorption in the form of a biofilm in the fermentation reactor, improving the volume-based productivity, is described below.

According to a first aspect, the abovementioned object, and also other advantages, are obtained by means of a process for producing alcohols, in which a sugary fluid is introduced into a fermentation reactor to produce a fermentation must enriched in isopropanol, butanol, ethanol and acetone relative to the sugary fluid, the fermentation reactor comprising a biomass produced by a strain belonging to the genus Clostridium which is supported (i.e. immobilized) on a solid support comprising a polyurethane foam.

According to one or more embodiments, the fermentation must comprises a supply of at least 0.2 g/L of isopropanol, at least 0.2 g/L of butanol, at least 0.2 g/L of ethanol and at least 0.2 g/L of acetone, relative to the sugary fluid. According to one or more embodiments, the fermentation must comprises a supply of at least 0.2 g/L of isopropanol, at least 0.2 g/L of butanol, less than 0.2 g/L of ethanol and at least 0.2 g/L of acetone, relative to the sugary fluid. According to one or more embodiments, the fermentation must comprises a supply of at least 0.2 g/L of isopropanol, at least 0.2 g/L of butanol, less than 0.2 g/L of ethanol and less than 0.2 g/L of acetone, relative to the sugary fluid. According to one or more embodiments, the fermentation must comprises a supply of at least 1 g/L of isopropanol, at least 2 g/L of butanol, relative to the sugary fluid. According to one or more embodiments, the fermentation must comprises a supply of at least 2 g/L of isopropanol, at least 4 g/L of butanol, relative to the sugary fluid. According to one or more embodiments, the fermentation must comprises a supply of at least 3 g/L of isopropanol, at least 6 g/L of butanol, relative to the sugary fluid. According to one or more embodiments, the fermentation must comprises a supply of at least 4 g/L of isopropanol, at least 8 g/L of butanol, relative to the sugary fluid. According to one or more embodiments, the fermentation must comprises a supply of at least 10 g/L of isopropanol, at least 20 g/L of butanol, relative to the sugary fluid. According to one or more embodiments, the fermentation must comprises a supply of at least 15 g/L of isopropanol, at least 30 g/L of butanol, relative to the sugary fluid. According to one or more embodiments, the fermentation must comprises a supply of at least 0.4 g/L of isopropanol+butanol, the isopropanol/(isopropanol+butanol) ratio possibly ranging from 0 to 1 (e.g. 0.01 to 0.99). According to one or more embodiments, the fermentation must comprises a supply of at least 3 g/L of isopropanol+butanol, the isopropanol/(isopropanol+butanol) ratio possibly ranging from 0 to 1 (e.g. 0.01 to 0.99). According to one or more embodiments, the fermentation must comprises a supply of at least 6 g/L of isopropanol+butanol, the isopropanol/(isopropanol+butanol) ratio possibly ranging from 0 to 1 (e.g. 0.01 to 0.99). According to one or more embodiments, the fermentation must comprises a supply of at least 9 g/L of isopropanol+butanol, the isopropanol/(isopropanol+butanol) ratio possibly ranging from 0 to 1 (e.g. 0.01 to 0.99). According to one or more embodiments, the fermentation must comprises a supply of at least 12 g/L of isopropanol+butanol, the isopropanol/(isopropanol+butanol) ratio possibly ranging from 0 to 1 (e.g. 0.01 to 0.99). According to one or more embodiments, the fermentation must comprises a supply of at least 30 g/L of isopropanol+butanol, the isopropanol/(isopropanol+butanol) ratio possibly ranging from 0 to 1 (e.g. 0.01 to 0.99). According to one or more embodiments, the fermentation must comprises a supply of at least 60 g/L of isopropanol+butanol, the isopropanol/(isopropanol+butanol) ratio possibly ranging from 0 to 1 (e.g. 0.01 to 0.99).

The production process according to the first aspect moreover makes it possible to maintain better stability of the microorganisms (e.g. maintenance of the performance qualities over time). Furthermore, although it is known that at and above a certain content in the fermentation medium, the fermentation products, and particularly the alcohols (e.g. butanol), have an inhibitory effect on the microorganism, the production process according to the first aspect makes it possible to at least partly reduce the negative effects of these inhibitory products on the biological activity present in the medium.

According to one or more embodiments, the sugary fluid is introduced continuously into the fermentation reactor.

According to one or more embodiments, the polyurethane foam comprises at least one of the following features:
volume cavities (i.e. pores or cells) whose equivalent sphere diameter is between 0.1 and 5 mm, preferably between 0.25 mm and 1.1 mm, preferably between 0.55 and 0.99 mm, and
a bulk density (i.e. apparent mass per unit volume) measured in air of between 10 and 90 g/L, preferably between 10 and 80 g/L, preferably between 15 and 45 g/L, such as between 20 and 45 g/L.

According to one or more embodiments, the fermentation is performed at a dilution rate (defined as the ratio of the flow rate of feedstock (liquid volume of the sugary fluid) to be converted relative to the liquid volume of the fermentation reactor) of between 0.04 h⁻¹ and 1 h⁻¹, preferably between 0.08 h⁻¹ and 0.5 h⁻¹, such as between 0.12 h⁻¹ and 0.3 h⁻¹.

According to one or more embodiments, the fermentation reactor comprises between 10% and 90%, preferably between 20% and 50%, preferably between 20% and 40% (e.g. 25-30%) by apparent volume of solid support relative to the total volume of the fermentation reactor.

According to one or more embodiments, the solid support is at least partially immersed, preferably totally immersed, in the reaction medium. According to one or more embodiments, the solid support is traversed by natural or forced convection with a stream of sugary fluid (e.g. downward, upward or radial fluid circulation, optionally in forced convection (e.g. Rushton-type radial turbomixer or axial turbomixer or through a support)).

According to one or more embodiments, the fermentation reactor is a reactor with upward or downward or radial fluid circulation and optionally with counter-current evolution of gas.

According to one or more embodiments, the biomass is produced by (and/or comprises) a microorganism belonging to the genus Clostridium and capable of producing mixtures of IBEA type (e.g. Clostridium acetobutylicum, Clostridium beijerinckii, Clostridium saccharobutylicum, Clostridium tyrobutyricum, C. saccharoperbutylacetonicum, C. butylicum and other Clostridium sp). The microorganism employed may or may not be genetically modified. More globally, the majority of the “solvent-producing” Clostridium species may be used. The strains preferably employed are those belonging to the species C. beijerinckii or C. acetobutylicum. They may or may not be genetically modified strains. A genetically modified strain corresponds to a strain whose genetic material (DNA) has been modified relative to an initial strain. The genetic modifications are made using genetic tools that are well known to those skilled in the art (cf. Pyne et al. Biotech Adv. 2014 32(3):623-41 and Wasels et al. J. Microbiol. Methods 2017 140:5-11). The genetic modifications may correspond to modifications of the actual genome content of the strain employed so as to improve its performance qualities for the production of Isopropanol/Butanol/Ethanol or its capacity to modify the selectivity toward isopropanol or n-butanol. The genetic modifications may also correspond to the integration of one (or more) genetic material(s) for improving the performance qualities or the selectivity toward isopropanol or n-butanol of the Clostridium strains used in the process. The term “genetic material” means a DNA fragment containing one or more genetic elements (promoter, gene, terminator, regulating structure, etc.) integrated into the genome of the genetically modified strain (cf. review by Walther & Francois Biotechnology Advances 34 (2016) 984-996 as regards Clostridium acetobutylicum). According to one or more embodiments, the biomass produced by the strain belonging to the genus Clostridium comprises a bacterium which may or may not be genetically modified belonging to the species Clostridium beijerinckii and/or Clostridium acetobutylicum.

According to one or more embodiments, the sugary fluid comprises an aqueous solution of C5 and/or C6 sugars obtained from lignocellulose, and/or of sugars obtained from sugar-producing plants (e.g. glucose, fructose and sucrose), and/or of sugars obtained from starchy plants (e.g. dextrins, maltose and other oligomers, or even starch). According to one or more embodiments, the aqueous solution comprises from 20 to 800 g/L (e.g. from 20 to 500 g/L) of sugar.

According to one or more embodiments, the sugary fluid is produced from a biomass feedstock. According to one or more embodiments, the biomass feedstock originates from the treatment of a renewable source. According to one or more embodiments, the renewable source comprises the lignocellulosic biomass (e.g. ligneous substrates, such as coniferous plants and deciduous plants (for example coniferous plants such as spruces or pines, or deciduous plants such as eucalyptus trees), agricultural byproducts (e.g. straw) or byproducts from industries generating lignocellulosic waste (agrifood and paper industries)), and/or plants of dedicated cultures (e.g. miscanthus, Panicum virgatum (switchgrass)), and/or sugar-producing plants, such as sugar beet, sugarcane, Jerusalem artichoke and/or starchy plants (e.g. corn and wheat) and/or tubers (e.g. cassava, Jerusalem artichoke and potato). According to one or more embodiments, the renewable source also comprises a lignocellulosic biomass of products and residues from the paper industry and lignocellulosic material transformation products.

According to one or more embodiments, the biomass feedstock comprises about 35% to 50% by weight of cellulose, 20% to 30% by weight of hemicellulose and 15% to 25% by weight of lignin relative to the total weight of the biomass feedstock.

According to one or more embodiments, the solid support is placed inside the fermentation reactor before and/or after immobilization (e.g. by adsorption) of the biomass produced by the strain belonging to the genus Clostridium on the solid support.

According to one or more embodiments, the biomass produced by the strain belonging to the genus Clostridium is immobilized on the solid support in a secondary tank, and the solid support supporting the biomass produced by the strain belonging to the genus Clostridium (also referred to hereinbelow as the bacterial biomass) is introduced into the fermentation reactor. It is possible to immobilize the bacterial biomass inside a secondary tank functioning in a rapid loop (“in stream”) relative to the fermentation reactor. It is notably possible to implement a batch step in the process of the present description, for example for a period of 4-5 hours, for example before continuously introducing sugary fluid into the fermentation reactor. According to one or more embodiments, the reaction medium which contains/submerges the solid support undergoes a degree of inoculation with a bacterial biomass inoculation solution (e.g. with cells at the substantially maximal growth rate) of between 0.5% and 20% by volume, preferably between 1% and 20% by volume relative to the total volume of the reaction volume (e.g. 10% by volume). According to one or more embodiments, the solid support is at least partially immersed, preferably totally immersed, in the inoculation solution during said “batch” step. According to one or more embodiments, the bacterial biomass is immobilized on the solid support in the form of a biofilm (i.e. a microbial group which may be either attached to a solid surface (organic or inorganic) or form flocs or aggregates in an isolated manner, notably by self-granulation, on the solid surface).

According to one or more embodiments, the solid support is introduced into the fermentation reactor in the form of one or more blocks. According to one or more embodiments, the solid support (e.g. in the form of a single block) is substantially in the form of a straight circular cylinder with a diameter less than or substantially equal to the inside diameter of the fermentation reactor. According to one or more embodiments, the block has a diameter substantially equal to the inside diameter of the fermentation reactor. Although the block and the fermentation reactor are described here as straight circular cylinders, it is understood that the block and the fermentation reactor may be of any shape. According to one or more embodiments, the block is centered or eccentric relative to the main axis (vertical) of the fermentation reactor, or next to a radial wall of the fermentation reactor.

According to one or more embodiments, the solid support is introduced into the fermentation reactor in the form of a net or of a latticed container comprising a plurality of polyurethane foam cubes, parallelepipeds or any other three-dimensional shapes (chips), for example of which at least one dimension is at least 3 mm. According to one or more embodiments, the net or the latticed container is a straight circular cylinder with a diameter less than or substantially equal to the inside diameter of the fermentation reactor. According to one or more embodiments, the net or the latticed container has a diameter substantially equal to the inside diameter of the fermentation reactor. Although the net, the latticed container and the fermentation reactor are described here as straight circular cylinders, it is understood that the net, the latticed container and the fermentation reactor may be of any shape.

According to one or more embodiments, the solid support forms a fluidized bed or a fixed bed in the fermentation reactor. According to one or more embodiments, the solid support forms a fluidized bed which is kept immersed, at least partially and preferably totally, by a grate.

According to one or more embodiments, the solid support is stirred (e.g. mechanically). According to one or more embodiments, the solid support is immobilized inside a net or a latticed container which is concentric, for example concentric to the axis of stirring (e.g. use of a Robinson-Mahoney type reactor).

According to one or more embodiments, the solid support (e.g. block(s), net or latticed container) is placed in the fermentation reactor so as to be flush with the surface of the reaction medium. According to one or more embodiments, the sugary fluid is introduced directly above or below the block(s), the net or the lattice.

According to one or more embodiments, the fermentation is anaerobic fermentation (e.g. strict), such as under a supply of inert gas (e.g. under nitrogen).

According to one or more embodiments, the fermentation is performed at a temperature of between 28° C. and 40° C., preferably between 30° C. and 37° C. (e.g. 36° C.), and/or at a pressure of between about 0.1 MPa and 0.15 MPa (i.e. atmospheric pressure+the water heads).

According to one or more embodiments, the fermentation is performed continuously for a time of at least 250 hours, preferably at least 500 hours without any upper limitation (e.g. 5000 hours).

According to a second aspect, the abovementioned subject, and also other advantages, are obtained by a fermentation reactor (2) comprising a biomass produced by a strain belonging to the genus Clostridium supported on a solid support (9) comprising a polyurethane foam.

According to one or more embodiments, at least a portion of the fermentation must obtained at the outlet of the fermentation reactor is recycled into the inlet of the fermentation reactor. It is notably possible to achieve linear speeds that are independent of the overall residence time.

Embodiments of the process referenced above and also other features and advantages of said process will emerge on reading the description that follows, which is given purely for nonlimiting illustrative purposes, and with reference to the following drawings.

### LIST OF FIGURES

FIG. 1 is a schematic view of a process for producing alcohols according to embodiments of the present description.

FIG. 2 is a schematic view of a solid support whose diameter is substantially equal to the inside diameter of a fermentation reactor according to embodiments of the present description.

FIG. 3 is a schematic view of solid supports that are centered, eccentric or next to the walls of fermentation reactors according to embodiments of the present description.

FIG. 4 is a schematic view of solid supports according to embodiments of the present description, comprising elements made of polyurethane foam confined in a net or a latticed container.

FIG. 5 is a schematic view of a solid support forming a fluidized bed contained immersed in a fermentation reactor according to embodiments of the present description.

FIG. 6 is a schematic view of a process for producing alcohols according to embodiments of the present description also comprising a fermentation finishing step.

FIG. 7 shows the change in volume productivity of IBEA of a reference process as a function of the imposed dilution rate.

### DESCRIPTION OF THE EMBODIMENTS

Embodiments of the process according to the first aspect and of the reactor according to the second aspect will now be described in detail. In the following detailed description, many specific details are presented in order to provide a more in-depth comprehension of the process. However, it will be apparent to a person skilled in the art that the process can be performed without these specific details. In other cases, well-known features have not been described in detail in order to avoid unnecessarily complicating the description.

In that which follows, the term “comprise” is synonymous with (means the same thing as) “include” and “contain”, and is inclusive or open and does not exclude other unspecified elements. In addition, in the present description, the terms “approximately”, “substantially”, “essentially” and “about” are synonymous with (mean the same thing as) a margin of greater and/or less than 10% of the given value.

The Sugary Fluid

According to one or more embodiments, the sugary fluid comprises an aqueous solution of C5 and/or C6 sugars obtained from lignocellulose, and/or of sugars obtained from sugar-producing plants (e.g. glucose, fructose and sucrose), and/or of sugars obtained from starchy plants (e.g. dextrins, maltose and other oligomers, or even starch). According to one or more embodiments, the aqueous solution of C5 and/or C6 sugars originates from the treatment of a renewable source. According to one or more embodiments, the renewable source is of the lignocellulosic biomass type which may notably comprise ligneous substrates (e.g. deciduous plants and coniferous plants), agricultural byproducts (e.g. straw) or byproducts from industries generating lignocellulosic waste (e.g. agrifood or paper industries). The renewable source may also originate from sugar-producing plants, for instance sugar beet and sugarcane, or from starchy plants such as corn and wheat. The aqueous solution of C5 and/or C6 sugars may also originate from a mixture of various renewable sources.

The Biomass Produced by the Strain Belonging to the Genus Clostridium

The bacterial biomass is mainly adsorbed in the form of a biofilm onto a solid support. Preferably, the bacteria are strains belonging to the species Clostridium beijerinckii and/or Clostridium acetobutylicum. The bacteria used in the process may be strains which may or may not be genetically modified and which naturally produce isopropanol and/or Clostridium strains which naturally produce acetone and which are genetically modified to make them produce isopropanol.

The Solid Support

The solid support comprises a polyurethane foam. Polyurethane foam is particularly advantageous since it allows access not only to the production of mixtures of IBEA type, but also allows access to production of continuous type by immobilization of the bacterial biomass. Specifically, the Applicant has demonstrated that polyurethane foam is capable of fixing bacteria of the genus Clostridium in a sufficiently substantial manner (i.e. beyond the dilution rate causing cell wash-out) making it possible to continuously produce mixtures of IBEA type. Furthermore, polyurethane foam is suitable for immobilization by immersion in a reactor.

According to one or more embodiments, the polyurethane foam has:
volume cavities (i.e. pores or cells) whose equivalent sphere diameter is between 0.1 and 5 mm, preferably between 0.25 mm and 1.1 mm, preferably between 0.55 and 0.99 mm, and/or
a bulk density (i.e. apparent mass per unit volume) measured in air of between 10 and 90 g/L, preferably between 10 and 80 g/L, preferably between 15 and 45 g/L, such as between 20 and 45 g/L or between 25 and 45 g/L.

Description of the Method for Measuring the Pore Diameter: X-Ray Tomography

The equivalent sphere diameter of the volume cavities may notably be obtained by analysis with an X-ray microscanner (e.g. HR 70 kV 200 microA Point focal medium tube; Varian pixel detector: 6 microns; acquisition time: 2 hours) of a sample (e.g. 7 mm×7 mm×15 mm) and reconstruction of a representative volume of the foam (e.g. reconstructed volume 5 mm×5 mm×5 mm with a voxel size of 6 microns), assuming spherical cells.

The diameter measurements were made by 3D image analysis with the Avizo software from 3D volumes acquired with an X-ray microscanner. The cells were artificially closed by image analysis so as to estimate the volume and then the diameter thereof. The diameter of a given cell is likened to that of a sphere of the same volume. The various steps of the image analysis are as follows:
thresholding of the images (black=cells and white=walls);
2D partitioning of the “cells” by the xy catchment basin method on Avizo;
3D partitioning of the cells by the 3D catchment basin method on Avizo;
removal of the edge cells (incomplete cells);
measurement of the volumes of the reconstructed cells;
estimation of the cell diameters (diameters of equivalent spheres of the same volume); and
size distribution of the cells for comparison.

The Process

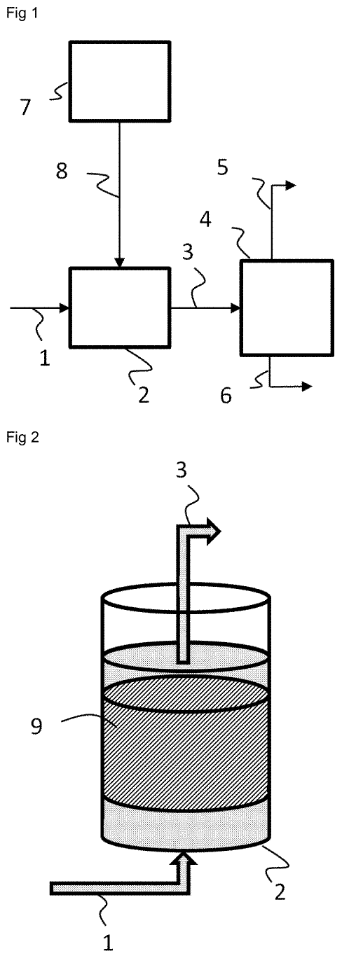

FIG. 1 shows a scheme for producing an alcohol mixture from a substrate of lignocellulosic biomass type.

With reference to FIG. 1, the sugary fluid comprising, for example, C5 and/or C6 sugars is introduced via line 1 into a fermentation reactor 2 to undergo a fermentation step.

In the fermentation reactor 2, the sugary fluid is placed in contact with the bacterial biomass supported on a solid support comprising a polyurethane foam. The fermentable sugars (e.g. C5 and/or C6 sugars) are thus transformed into alcohols and/or solvents by the microorganisms to produce a (first) fermentation must (or liquor or wine), which is notably enriched in isopropanol, butanol, ethanol and acetone relative to the sugary fluid.

The fermentation step in the fermentation reactor 2 may be performed at a temperature of between 28° C. and 40° C., preferably between 30° C. and 37° C., so that the fermentation must comprises fermentation reaction products of IBEA type, for example isopropanol, which is then evacuated via a line 3.

The fermentation must is introduced via line 3 into a separation unit 4 (optional) for separating and extracting the compounds of interest from the fermentation must, said compounds being removed via line 5. The separation residues, commonly known as vinasses, are removed from the separation unit 4 via line 6. The vinasses are generally composed of water and also of any liquid or solid product not converted or extracted during the preceding steps. The separation unit 4 may implement one or more distillations, and optionally a separation of the solid matter and/or the matter in suspension, for example by centrifugation, decantation and/or filtration.

Several fermentation reactor implementations or technologies existing in the prior art are suitable for immobilizing the bacterial biomass by adsorption on the solid support, and can do so whether the implementation takes place inside the fermentation reactor 2 or in a secondary tank in “in-stream” mode. For example, the bacterial biomass may be immobilized on a solid support directly in the fermentation reactor 2 or indirectly in a secondary tank 7 (optional), for example functioning in “in-stream” mode relative to the fermentation reactor 2. The solid support thus loaded with bacterial biomass may then be introduced into the fermentation reactor, for example via line 8 or via any other means.

According to one or more embodiments, the solid support forms a fluidized bed or a fixed bed.

In the case of a fluidized bed, a stream of liquid may be established directly on the bed of polyurethane foam in the downward direction (since the density of the fermentation must is generally greater than the density of the polyurethane foam). According to one or more embodiments, the liquid surface speed of the sugary fluid is greater than the minimum fluidization speed. According to one or more embodiments, the liquid surface speed of the sugary fluid is modified (e.g. increased) as a function of the change in the density difference which takes place in the course of fermentation. For example, gradually as the biofilm forms on the solid support, the density of the solid support may come to vary (e.g. to increase), giving rise to evolutive hydraulic regimes (e.g. different optional recycling rates at the start of fermentation and at the end of fermentation).

In the case of a fixed bed, a solid support comprising a loose or structured stack of polyurethane foam particles may be envisaged, with or without mechanical stirring (e.g. inside a column).

According to one or more embodiments, the fermentation medium passes through the solid bed as an upflow or a downflow. For example, in the case of a downward liquid circulation, a system enabling counter-current evolution of gas may be provided. According to one or more embodiments, radial circulation may also be envisaged in the fermentation reactor 2, for example in the case where mechanical stirring is applied at the center of the fermentation reactor 2 (e.g. a Rushton-type radial turbomixer). According to one or more embodiments, the solid is immobilized inside a basket concentric to the axis of stirring, notably making it possible to control the speeds of the reaction medium and the hydrodynamics imposed around said medium (e.g. Robinson-Mahoney type reactor).

Immersion and Use of the Solid Support

According to one or more embodiments, the solid support is partially or totally immersed, in order notably to increase the formation of the biofilms and to improve the performance.

According to one or more embodiments, the solid support is introduced in the form of a single block, for example in the form of a cylinder with a diameter less than or substantially equal to the inside diameter of the fermentation reactor 2. According to one or more embodiments, as shown in FIG. 2, the diameter of the solid support 9 is substantially equal to the inside diameter of the fermentation reactor 2. The block may thus correspond to a filter medium inside which the biofilms grow. Although it is possibly more difficult to control a supply of insoluble materials that may be entrained with the sugary fluid, the performance qualities of a process using a fermentation reactor 2 as presented in FIG. 1 are among the highest. On the other hand, the solid support 9 as shown in FIG. 2 may give rise to a slight positive pressure in the free liquid phase, at the lower level. It should be noted that if the evolution of gas is substantial during the fermentation, a gas pocket may form under the solid support and preferential passages inside the solid support 9 may be generated during the evacuation of the gas.

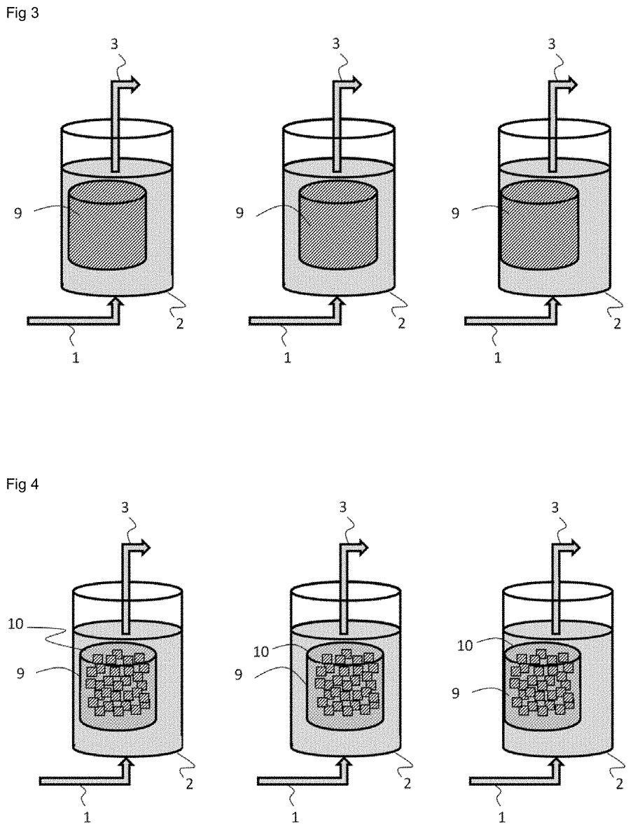

According to one or more embodiments, as shown in FIG. 3, the block of solid support 9 may be centered, eccentric or next to a wall of the fermentation reactor 2. Advantageously, the solid support 9 does not in any way disrupt the circulation of the liquid at the inlet or at the outlet of the process, notably when performed continuously. Furthermore, the possible presence of insoluble materials such as those derived from the major cereals does not pose any problems. The stream of sugary fluid arriving via line 1 may also be introduced at the level of the blocks of solid support 9, for example when they are flush with the surface of the reaction medium of the fermentation reactor 2. Advantageously, when the solid support is flush with the surface of the reaction medium at the level of the inlet of the sugary fluid, the medium is locally less concentrated in alcohol and growth of the bacteria is promoted.

It is understood that it is not required to use a monobloc solid support 9. According to one or more embodiments, the solid support comprises a net or a latticed container 10 comprising cubes or parallelepipeds or other three-dimensional elements of any shape (e.g. polyhedra) of large or small size (e.g. at least one dimension between 3 mm and 10 m, such as from 2 cm to 1 m), as shown in FIG. 4, the elements being constituted of polyurethane foam. According to one or more embodiments, the net or the latticed container 10 forms a cylinder whose diameter is less than or substantially equal to the inside diameter of the fermentation reactor 2. According to one or more embodiments, the net or the latticed container 10 has a diameter substantially equal to the inside diameter of the fermentation reactor 2.

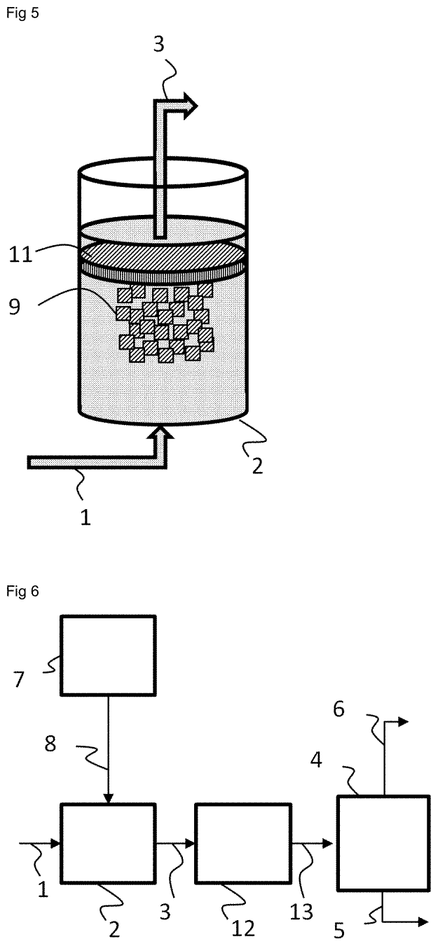

It may arise that evolutions of gas have a tendency to make the solid support 9 rise. On the other hand, a perforated plate, a simple net or a grate 11 (see FIG. 5) may suffice to keep the solid support, for example in movement, in the fermentation reactor 2.

In the embodiments of FIGS. 2 to 5, the fermentation reactor 2 has upward fluid circulation. On the other hand, it is understood that the direction of circulation of the sugary fluid may be downward. It is also understood that the direction of circulation may be globally upward or downward (viewed from outside the fermentation reactor) and radial inside the fermentation reactor 2.

Finishing Steps

As indicated in FIG. 6, the alcohol production process may implement a finishing reactor 12 known as a “finisher” (optional). According to one or more embodiments, the fermentation must withdrawn from the fermentation reactor 2 via line 3 is introduced into the finishing reactor 12 suitable for producing a second fermentation must enriched in IBEA relative to the fermentation must withdrawn from the fermentation reactor 2. The second fermentation must is then withdrawn from the finishing reactor 12 and evacuated via line 6 and introduced into the separation unit 4 (optional) for separating and extracting the compounds of interest from the fermentation must, said compounds being removed via line 5. The finishing reactor 12 is preferably without PU foam. The finisher is preferably homogeneous and has the purpose of optimally exploiting the IBEA titer potential of the Clostridium strain, by enabling a prolonged residence time, and of adding an amount of carbon-based substrate which meets the needs of the strain. The finishing reactor 12 notably makes it possible to ensure depletion of the saccharides.

According to one or more embodiments, the alcohol production process involves a step of partial recovery of the IBEA compounds produced which are present in the first and/or second fermentation must. This step of extracting the IBEA compounds involves a step of stripping with a pressurized gas sent into the fermentation reactor 2 and/or the finishing reactor 12 so as to entrain the alcohols present in the aqueous phase.

According to one or more embodiments, the stripping gas is a gas produced directly by fermentation and which has been stored beforehand (via methods known to those skilled in the art) before its use. The stripping gas typically comprises carbon dioxide and possibly hydrogen. This step of stripping with gas advantageously makes it possible to control during the fermentation the content of alcohols present in the medium so as to limit the inhibition of the microorganisms which arises when the alcohol content reaches a critical value. According to one or more embodiments, the step of stripping with gas may be performed either continuously or batchwise. The flow rate of fermentation gas relative to the fermenter volume is, for example, between 0.5 and 2.5 I/1/min and preferably between 0.7 and 1.1 I/1/min.

According to one or more embodiments, the recovery process may also be performed so that the step of stripping with gas is performed in a finisher 12 containing a water-immiscible organic solvent, the solvent forming a supernatant organic phase above the fermentation must. The solvent will moreover be chosen so as to be biocompatible with the microorganism.

The stripping gas is thus injected into the fermentation must so as to entrain the alcohols produced into the supernatant organic phase and so that a portion of the alcohols is transferred into the organic phase when the stripping gas passes through said organic phase.

### EXAMPLES

Reference Example: Continuous Test of Cells in Suspension

A continuous test with cells in suspension is performed experimentally. A bioreactor with a total volume of 5 L is filled with 1.8 L of fermentation medium. The initial glucose is set at 60 g/L, and the inoculum is 0.2 L, i.e. an inoculation rate of 10% by volume relative to the total volume of the fermentation medium with cells at the maximum growth rate, after having been purged with nitrogen for 1 hour for the purpose of ensuring (strict) anaerobic conditions from the start of the test. Purging with nitrogen is maintained during the preliminary batch step (4-5 hours). The microorganism employed is Clostridium beijerinckii DSM 6423. The temperature and the mechanical stirring are set at 36° C. and 60 rpm, respectively, from the start of the test; the pressure is substantially atmospheric+the water head of the bioreactor. The test is performed in 2 steps:
a first batch step (duration of 4-8 hours) corresponding to the lag time and start of the exponential growth (accompanied by generation of fermentation gases); and
a second continuous-mode step in which different dilution rates are imposed.

A period of time corresponding to at least three times (preferably at least five times) the residence time is allowed to pass at each new nominal value for the purpose of stabilization.

Next, the glucose and main metabolites (i.e. isopropanol, butanol, ethanol and acetone) of the fermentation must are analyzed.

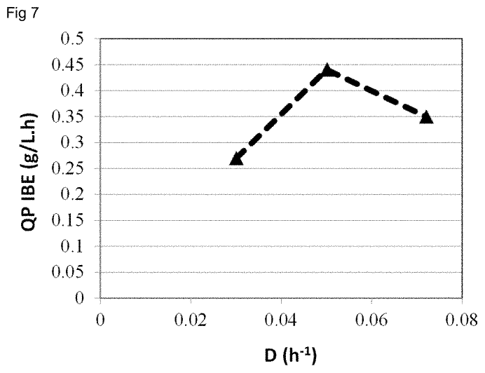

FIG. 7 shows the change in volume productivity r of IBEA g/L.h as a function of the dilution rate D imposed in the bioreactor (h⁻¹). The dilution rate D is defined as the volume flow rate entering the reactor divided by the reactor volume. In the case of a process with microorganisms in suspension, this parameter may be considered simultaneously as the inverse of the residence time for the fluid and for the microorganisms. Consequently, cell wash-out appears above a certain dilution rate, leading to a loss of volume productivity. In this case, the critical dilution rate is between 0.04 and 0.06 h⁻¹, at which moment the maximum productivity reaches a value of about 0.45 g/L.h of IBEA.

### Example 1 According to the Present Description: Test in Batch Mode with Polyurethane Foam

A process in batch mode is performed experimentally: two fermentations with solid supports of polyurethane foam type (Foam 1, Foam 2) having different physical and structural characteristics, and a control fermentation (cells in suspension). The main characteristics of these two foams are presented in table 1 below:

TABLE 1

| Solid support | Foam 1 | Foam 2 |
|---|---|---|
| Pore diameters (mm) | 0.73 | 1 |
| Bulk density (g/L) | 29.8 | 20.5 |

Several bioreactors are filled with 20 mL of fermentation medium, which has been placed beforehand under anaerobic conditions in order to ensure the absence of oxygen. 40% of solid volume (apparent volume) relative to the total volume of the bioreactor are introduced into each bioreactor. The initial glucose is set at 90 g/L, the seeding rate is 10% (liquid volume) (same inoculum for all the fermentations) with cells at the maximum growth rate. The microorganism employed is Clostridium beijerinckii DSM 6423. The bioreactors are all placed in an anaerobic jar and at the set temperature (36° C.) for a set period of 12 days. The pressure is substantially atmospheric+the water head of the bioreactor. Next, the final fermentation must is analyzed, as are the solids supporting the biofilm. Each operating condition is performed in triplicate so as to ensure the repeatability of the experiments.

The presence of the solids of the polyurethane foam type has several effects on the consequent overconsumption of glucose (see table 2 below) and on the final IBEA titer.

TABLE 2

| | Foam | Foam |
|---|---|---|
| Solid support | 1 | 2 |
| Glucose consumption (g/L) | 51.4 | 44.7 |
| Overconsumption of glucose (%) (relative | 31.4 | 14.3 |
| to the control which has no solid support | | |
| according to the present description) | | |

The titer is quantified from the fermentation yield which is considered invariable and the glucose consumption on each test. Thus, the batch fermentations with Foam 1 and Foam 2 produced, respectively, 17.5 g/L and 15.2 g/L of IBEA, the titer in both cases being greater than that obtained with the control fermentation (13.3 g/L of IBEA).

### Example 2 According to the Present Description: Continuous Test with Polyurethane Foam

The process is performed experimentally according to embodiments of the present description, using two fermentations in continuous mode with immobilization on solid supports of polyurethane foam type (Foam 1, Foam 2) having different physical and structural characteristics. The main characteristics of these two foams are presented in table 1. The packing is performed in loose mode, with cubes 3 mm×5 mm×5 mm in size.

The solid support is introduced into a 112 ml glass column (dimensions: inside diameter=32 mm, H=13.9 cm), to a volume proportion of 50% in the column relative to the total internal volume of the column. The column is placed in the recirculation loop of a second fermentation flask, of which the volume of fermentation medium reaches 80 ml. The entire fermentation system was placed beforehand under anaerobic conditions by purging with nitrogen so as to ensure the absence of oxygen. The glucose in a feed tank is set at 90 g/L, and the solid support undergoes an inoculation rate of 10% by volume with cells at the maximum growth rate relative to the total volume of the fermentation medium. The microorganism employed is Clostridium beijerinckii DSM 6423. The temperature of the system is set (36° C.), without stirring. The pressure is substantially atmospheric+the water head of the glass column. The test is performed in 2 steps:
a first batch step (duration of 4-5 hours) corresponding to the “lag time” and start of the exponential growth (accompanied by generation of fermentation gases); and
a second continuous-mode step in which different dilution rates are imposed.

A period of time corresponding to at least three times the residence time is allowed to pass at each new nominal value for the purpose of stabilization. Periodically, the final fermentation must is collected under sterile conditions from the column and the glucose and main metabolites (i.e. isopropanol, butanol, ethanol and acetone) are analyzed.

The presence of a solid support of the polyurethane foam type has several effects on the fermentation in continuous mode: table 3 below shows the evolution of the fermentation, as a function of the imposed dilution rate, of the percentage of glucose consumption (%), of the total content of IBEA (g/L) and of the volume productivity (g/L.h of IBEA). Relative to the conditions of cells in suspension (reference example), an increase in productivity is observed in the case where the cells are immobilized on Foam 1 (factor 6) or Foam 2 (factor 3).

TABLE 3

| | Foam 1 | Foam 2 | Free cells |
|---|---|---|---|
| Dilution rate (h⁻¹) | 0.14 | 0.14 | 0.05 |
| Glucose consumption (g/L) | 48.6 | 23.4 | 28.8 |
| Content of IBEA (g/L) | 17 | 9 | 9 |
| Volume productivity (g/L · h) | 2.46 | 1.3 | 0.4 |

Although embodiments and examples mentioned above are described in detail, it is understood that additional embodiments may be envisaged. For example, the bacterial biomass used in the process according to the present description may correspond to a strain other than Clostridium beijerinckii DSM 6423. Also, the polyurethane foams according to the present description may be other than those described in table 1. Furthermore, improved IBEA contents and volume productivities may be obtained by means of inoculation rates, sugar contents, dilution rates, temperatures, pressures, stirring conditions, durations, etc. other than those indicated in the examples.

Unless otherwise mentioned in the present description, it will be apparent to a person skilled in the art that all the embodiments described above may be combined together. For example, unless otherwise specified, all the characteristics of the embodiments described above may be combined with or replaced with other characteristics of other embodiments.

### Example 3 According to the Present Description: Continuous Test with Polyurethane Foam

The process is performed experimentally according to embodiments of the present description, using two fermentations in continuous mode with immobilization on solid supports of polyurethane foam type. The packing is performed in loose mode, with cubes 10 mm×10 mm×7 mm in size. The foams have a macropore size of about 1 mm.

The two fermenters are in the form of a glass column with a working volume of 250 ml. The fermenter packing conditions are presented in table 4.

TABLE 4

| | Fermenter 1 | Fermenter 2 |
|---|---|---|
| Mass of foams (g) | 2.03 | 3.04 |
| Working volume of liquid (mL) | 250 | 250 |
| Bulk density (g/L) (%) | 37 | 37 |

A recirculation loop is placed between the inlet and the outlet of the reactor to maintain good homogenization in the fermenter. The liquid outlet takes place by overflow. The entire fermentation system was placed beforehand under anaerobic conditions by purging with nitrogen so as to ensure the absence of oxygen. The glucose concentration in the feed tank is set at 60 g/L. The fermenter is inoculated to 10% by volume with cells at the maximum growth rate relative to the total volume of the fermentation medium. The microorganism employed is Clostridium beijerinckii DSM 6423. The temperature of the system is set at 34° C. without stirring other than the recirculation. The pressure is substantially atmospheric.

The test is performed in 2 steps:
a first batch step (duration of 7 hours) corresponding to the “lag time” and start of the exponential growth (accompanied by generation of fermentation gases); and
a second continuous-mode step in which different dilution rates are imposed.

In the second step, the dilution rate is increased continuously as a function of the solvent concentrations. Periodically, the fermentation must is collected under sterile conditions and the glucose and main metabolites (i.e. isopropanol, butanol, ethanol and acetone) are analyzed. The pH is also measured.

The two fermenters are run for a period of 912 hours. The dilution rate ranges between 0.02 h⁻¹ and 0.23 h⁻¹. After 200 hours of fermentation, the total content of IBEA ranges between 8 and 16 g/L. Before 500 hours, the maximum volume productivity for the two reactors is 1.5 g/L.h of IBEA. The best performance on the entire test gives a maximum volume productivity of 2.44 g/L/h of IBEA for fermenter 1 and 2.24 g/Uh of IBEA for fermenter 2.

### Example 4 According to the Present Description: Continuous Test with Polyurethane Foam in a Stirred Reactor

A continuous-mode test is performed experimentally with cells immobilized on a support of polyurethane foam type. A bioreactor with a total volume of 5 L is filled with 2 L of fermentation medium. The initial glucose is set at 60 g/L, and the inoculum is 0.2 L, i.e. an inoculation rate of 10% by volume relative to the total volume of the fermentation medium with cells at the maximum growth rate, after having been purged with nitrogen for 1 hour for the purpose of ensuring (strict) anaerobic conditions from the start of the test. Purging with nitrogen is maintained during the preliminary batch step (7 hours). The microorganism employed is Clostridium beijerinckii DSM 6423. The temperature is set at 34° C. The mechanical stirring ranges between 60 and 170 rpm. The pressure is substantially atmospheric+the water head of the bioreactor.

The test is performed in 2 steps:
a first batch step (duration of 7 hours) corresponding to the “lag time” and start of the exponential growth (accompanied by generation of fermentation gases); and
a second continuous-mode step in which different dilution rates are imposed.

In the second step, the dilution rate is increased continuously as a function of the solvent concentrations. Periodically, the fermentation must is collected under sterile conditions and the glucose and main metabolites (i.e. isopropanol, butanol, ethanol and acetone) are analyzed. The pH is also measured.

The two fermenters are run over a period of about 765 hours. The dilution rate ranges between 0.02 h⁻¹ and 0.2 h⁻¹. After 100 hours, the total content of IBEA ranges between 5 and 16 g/L. The maximum volume productivity obtained for this fermentation is 1.6 g/L.h of IBEA.

1. A process for producing alcohols, in which a sugary fluid is introduced into a fermentation reactor (2) to produce a fermentation must enriched in isopropanol, butanol, ethanol and acetone relative to the sugary fluid, the fermentation reactor (2) comprising a biomass produced by a strain belonging to the genus Clostridium which is supported on a solid support (9) comprising a polyurethane foam.

2. The process as claimed in claim 1, in which the polyurethane foam has: volume cavities in which the equivalent sphere diameter is between 0.1 and 5 mm; and/or a bulk density measured in air of between 10 and 90 g/L.

3. The process as claimed in claim 1, in which the sugary fluid is introduced continuously into the fermentation reactor (2).

4. The process as claimed in claim 3, in which the fermentation is performed at a dilution rate (D) of the sugary fluid relative to the volume of the fermentation reactor (2) of between 0.04 h⁻¹ and 1 h⁻¹.

5. The process as claimed in claim 1, in which the biomass produced by the strain belonging to the genus Clostridium comprises a bacterium belonging to a species selected from the species Clostridium acetobutylicum, Clostridium beijerinckii, Clostridium saccharobutylicum, Clostridium tyrobutyricum, C. saccharoperbutylacetonicum, C. butylicum.

6. The process as claimed in claim 1, in which the biomass produced by the strain belonging to the genus Clostridium comprises a genetically modified bacterium belonging to the species Clostridium beijerinckii which naturally produces isopropanol and/or Clostridium acetobutylicum which is genetically modified for the production of isopropanol.

7. The process as claimed in claim 1, in which the biomass produced by the strain belonging to the genus Clostridium is immobilized on the solid support (9) in a secondary tank (7), and the solid support (9) supporting the biomass produced by the strain belonging to the genus Clostridium is introduced into the fermentation reactor (2).

8. The process as claimed in claim 1, in which the biomass produced by the strain belonging to the genus Clostridium is immobilized on the solid support in the form of a biofilm.

9. The process as claimed in claim 1, in which the solid support (9) is introduced into the fermentation reactor (2) in the form of one or more blocks, and/or in the form of a net or a latticed container (10) comprising a plurality of polyurethane foam cubes, parallelepipeds or any other three-dimensional forms.

10. The process as claimed in claim 1, in which the solid support (9) is substantially in the form of a straight circular cylinder with a diameter less than or substantially equal to the inside diameter of the fermentation reactor (2).

11. The process as claimed in claim 1, in which the solid support (9) forms a fluidized bed or a fixed bed in the fermentation reactor (2).

12. The process as claimed in claim 1, in which the solid support (9) is placed in the fermentation reactor (2) so as to be flush with the surface of the reaction medium of the fermentation reactor (2).

13. The process as claimed in claim 1, which the production of the fermentation must is performed by anaerobic fermentation.

14. The process as claimed in claim 1, in which the fermentation is performed at a temperature of between 28° C. and 40° C., and/or at a pressure of between about 0.1 MPa and 0.15 MPa, and/or for a time of at least 250 hours.

15. A fermentation reactor (2) comprising a biomass suitable for producing isopropanol, butanol, ethanol and acetone and produced by a strain belonging to the genus Clostridium supported on a solid support (9) comprising a polyurethane foam.
